(19) United States
(12) Patent Application Publication
(10) Pub. No.: US 2014/0248651 A1
(43) Pub. Date: Sep. 4, 2014

(54) NUTRIENT MEDIUM

(71) Applicants: Heinz-Jürgen Lögtenbörger, Bickenbach (DE); Kay Fiebig, Darmstadt (DE)

(72) Inventors: Heinz-Jürgen Lögtenbörger, Bickenbach (DE); Kay Fiebig, Darmstadt (DE)

(73) Assignee: DOHLER GMBH, Darmstadt (DE)

(21) Appl. No.: 14/236,426

(22) PCT Filed: Aug. 1, 2012
(86) PCT No.: PCT/EP2012/065049

§ 371 Date: May 19, 2014

(30) Foreign Application Priority Data
Aug. 1, 2011 (EP) 11176196.1

(51) Int. Cl. C12N 1/16

(52) U.S. Cl. CPC C12N 1/16
USPC 435/29; 435/255.7

(57) ABSTRACT

The invention relates to a nutrient medium, containing 10 to 20 parts by weight of yeast extract, 15 to 30 parts by weight of peptone, 35 to 75 parts by weight of monosaccharides and disaccharides, up to 3 parts by weight of mineral substances, 0.02 to 1 parts by weight of gellan gum, and water.

The invention relates to a nutrient medium, and the use thereof.

In the production of a large part of goods, mainly consumer goods, of daily need, such as food products, for example, in the form of non-alcoholic beverages or beers, care must be taken to ensure that these are free from metabolizing living microorganisms, such as molds, yeasts, lactic or acetic acid bacteria, or that less than a maximum level thereof defined as tolerable in accordance with the corresponding products is present. Microbiological detection systems for this purpose have long been known.

Typical nutrient media employed for analytical purposes include gelled solid nutrient media based on, for example, agar, on which microorganisms can grow in the form of colonies.

A wide-spread method is based on the German Utility Model No. 93 19 470.6, in which a nutrient medium is described that is prepared by the user from dry individual components or a corresponding powder premixture, adjusted to a pH value of, for example, from 4.3 to 4.4, followed by pasteurization. During the production of the product to be examined, samples are taken from the production process and admixed with the nutrient medium. Subsequently, the growth of microorganisms, if present in a sample, can be monitored within a period of about 1 to 2 days in a light box at a temperature of about 28° C. If a sample becomes turbid, it has a load of microorganisms. At the same time, the degree of turbidity provides an indication of the extent to which the sample is loaded with microorganisms.

What is disadvantageous with the known method is the fact that the user has to prepare the nutrient medium, which is relatively time consuming and laborious. This requires that the user be experienced in dealing with microbiological nutrient media, and that the laboratory be correspondingly equipped with magnetic stirrers, Erlenmeyer flasks, pH meters with temperature compensation, precision scales, a pasteurization device, etc.

When the conditions are not observed, or there is contamination during the preparation, the corresponding nutrient medium could be obtained in an inferior quality, which could lead to difficulties when used, for example, false positive results.

It is the object of the present invention to overcome at least some of the drawbacks of the prior art.

This object is achieved by a liquid nutrient medium containing
10 to 20 parts by weight of yeast extract;
15 to 30 parts by weight of peptone;
35 to 75 parts by weight of mono- and disaccharides;
up to 3 parts by weight of minerals;
0.02 to 1 part by weight of stable gelling agent; and
water.

Water is preferably employed in an amount of from 100 to 2000, more preferably from 1000 to 2000, parts by weight. Preferably, the concentration of the stable gelling agent is ≦0.5% by weight, preferably ≦0.1% by weight, more preferably ≦0.05% by weight.

In contrast to the nutrient media of the prior art, the product can be prepared already by the manufacturer, and delivered to the customer in a ready-to-use form.

Surprisingly, the product with the composition according to the invention is stable over a period of more than six months when stored at room temperature, if it was autoclaved or, alternatively, subjected to ultra-high temperature processing.

In the usual manner, the nutrient medium according to the invention contains nutrients for microorganisms from the admixture of yeast extract, peptone, mono- and disaccharides, and minerals.

According to the invention, the nutrient medium contains one or more stable gelling agents, which are contained in an amount that leads to a flowable consistency of the nutrient medium. The gelling agent must be so stable that the product resists autoclavation, i.e., heating at a temperature of 121° C. for a holding time of at least 5 minutes, or industry standard ultra-high temperature (UHT) processing.

Usual flowable nutrient media contain, for example, carrageenan. However, under the existing conditions, carrageenan will not endure autoclavation and, therefore, is not a stable gelling agent within the meaning of the described invention. Preferably, gellan gum is employed as the gelling agent, but other natural, semi-synthetic or synthetic polymeric substances with gelling properties analogous to those of gellan gum may also be employed.

“Gelling properties analogous to those of gellan gum” means that the substances are capable of achieving stabilization and fixation of particles described in the solution without causing a significant build-up of viscosity. In particular, a stable gelling agent is one that, when used in the amounts mentioned, and after autoclavation with a holding time of 5 min at 121° C., still forms a system whose viscosity is above that of the same system without addition of the gelling agent, but is less than 100 mPa·s at 20° C.

Preferably, the viscosity of the system is at least 2 mPa·s.

Gellan gum is available, for example, from Merck and Co., U.S.A., under the designation of Gelrite. In the prior art, gellan gum is used in the presence of divalent ions, forming solid gels. In contrast, the nutrient media according to the invention are liquid.

Unstable gelling agents, such as carrageenan, are changed during autoclavation so as to lose their gelling properties. Although they may be pasteurized if necessary, this is not sufficient for long-term storability of the corresponding nutrient media, but it means that the nutrient media must subsequently be employed after only a short-term storage.

In the mentioned quantity ranges, liquids are obtained that preferably have a viscosity of <100, preferably <10, mPa·s at 20° C. In contrast to usual nutrient media, which are either compact or, as a broth, do not ensure fixation or holding together of a microorganism colony, the nutrient media according to the invention are flowable and provide for fixation (no sedimentation) and holding together of a growing colony (which is thus readily recognizable to the human eye). Nutrients can diffuse freely without being hindered by a matrix.

Because of the composition according to the invention, the nutrient media are storable for a long period of time after autoclavation or, alternatively, ultra-high temperature processing. When stored at 4 to 8° C. in a closed vessel in the dark, which is to be preferred, the properties of the product will not change over a period of twelve months.

While it is possible to deliver the nutrient medium in a dispensing bottle to the user, it is particularly preferred for the nutrient medium to be packaged in portions, for example, in vessels with transparent walls that can be hermetically sealed. For example, screw cap vessels are particularly suitable. What is left for the user to do is to open the screw cap vessel, to add the sample, and to close the screw cap vessel again.

For use, the nutrient medium is admixed with a sample and stored in an incubator, for example, at 25 to 37° C. The microorganisms can grow in the nutrient medium, which is shown by a turbidity of the medium, which is locally restricted in a minimum case. The extent of turbidity may at the same time be a measure of the germ load of the sample. In this way, a large number of samples can be simply divided “at first sight” into loaded and unloaded samples.

The use of the nutrient medium is suitable, above all, in fields where verification of the microbiological state of products that are consumed by living creatures or applied thereto in all conceivable dosage forms is considered important, for example, in the field of beverage and food production, fodder production, cosmetics or pharmaceutical industry. In addition, the nutrient medium in a form adapted from case to case is suitable for the field of clinical diagnostics.

In principle, the microorganisms can be obtained from the nutrient media simply by pipetting, and further characterized, for example, by means of PCR or similar methods.

The invention is further illustrated by the following non-limiting Examples:

### COMPARATIVE EXAMPLE 1

A nutrient medium was prepared with the following ingredients in the dry matter:
70% mono- and disaccharides
2% minerals
14% yeast extract
15.1% peptone
0.9% carrageenan.

Five parts of this dry matter was admixed with 95 parts of water, and after adjusting the pH to 4.3 and pasteurization at 80° C. for 5 minutes, the resulting detection medium was admixed with a highly diluted yeast suspension.

Incubating the screw cap vessel at 25° C. yields a result interpretable with the naked eye after 1 to 3 days, namely the growth of yeast cell colonies, which are recognizable as discrete punctiform turbidities freely floating in the detection medium.

### COMPARATIVE EXAMPLE 2

A similar method as in Comparative Example 1 was applied, except that the ready-to-use nutrient medium was not pasteurized, but autoclaved at 121° C. for 5 minutes.

Upon inoculation with yeast cells, the formation of a sediment caused by the microorganisms occurs after 1 to 3 days. A simple and telling interpretation of the microbiological result is not possible in this case without manipulation of the screw cap vessel.

### EXAMPLE 1

A nutrient medium deviating from that used in Comparative Examples 1 and 2 with respect to the gelling agent employed was prepared with the following ingredients in the dry matter:
70% mono- and disaccharides
2% minerals
14% yeast extract
15.1% peptone
0.9% gellan gum.

Five parts of this dry matter was admixed with 95 parts of water, and after adjusting the pH to 4.3 and autoclavation at 121° C. for 5 minutes, the resulting detection medium was admixed with a highly diluted yeast suspension.

Incubating the screw cap vessel at 25° C. yields a result interpretable with the naked eye after 1 to 2 days, namely the growth of yeast cell colonies, which are recognizable as discrete punctiform turbidities freely floating in the detection medium.

1. A liquid nutrient medium comprising
10 to 20 parts by weight of yeast extract;
15 to 30 parts by weight of peptone;
35 to 75 parts by weight of mono- and disaccharides;
up to 3 parts by weight of minerals;
0.02 to 1 part by weight of stable gelling agent; and
water.

2. The nutrient medium according to claim 1, wherein mono- and disaccharides are selected from maltose, dextrose, sucrose and fructose.

3. The nutrient medium according to claim 1, wherein 1 to 10 parts of lactic acid or salts thereof in equivalent molar amounts are additionally contained.

4. The nutrient medium according to claim 1, wherein said nutrient medium gel is storable at 4 to 8° C. for at least 365 days.

5. The nutrient medium according to claim 1, wherein said nutrient medium has been autoclaved or, alternatively, subjected to ultra-high temperature processing.

6. The nutrient medium according to claim 1, wherein said stable gelling agent is gellan gum.

7. The nutrient medium according to claim 1, wherein the nutrient medium has a viscosity at 20° C. is from 2 to 100 mPa·s.

8. The nutrient medium according to claim 1, comprising ≦0.5% by weight of stable gelling agents.

9. The nutrient medium according to claim 1, comprising ≦0.1% by weight of stable gelling agents.

10. Use of a nutrient medium according to claim 1, for microbiological screening, in the food industry, such as in the beverage industry, in the production of non-alcoholic beverages and bases thereof, in the animal fodder industry, in the cosmetics and pharmaceutical industries, and in the diagnostic field.

11. Use of a nutrient medium according to claim 10 for microbiological screening in the field of beer brewing.
